(19) United States
(12) Patent Application Publication
(10) Pub. No.: US 2026/0369352 A1
(43) Pub. Date: Nov. 19, 2026

(54) FREE-ENERGY-PERTURBATION (FEP) PATH GENERATION USING GENERATIVE ARTIFICIAL INTELLIGENCE MODELS

(71) Applicant: NVIDIA Corporation, Santa Clara, CA (US)

(72) Inventors: Yuxing PENG, Santa Clara, CA (US); Emine KUCUKBENLI, Melrose, MA (US); Guoqing ZHOU, Placentia, CA (US); Michelle Lynn GILL, New York, NY (US); Micha LIVNE, Austin, TX (US); Maria KORSHUNOVA, Sunnyvale, CA (US); Timur RVACHOV, Brooklyn, NY (US); Yonatan ISRAELI, Los Altos, CA (US)

(21) Appl. No.: 19/208,119

(22) Filed: May 14, 2025

(51) Int. Cl. G16B 20/30; G16B 40/00; G16B 50/30

(52) U.S. Cl. CPC G16B 20/30; G16B 40/00; G16B 50/30

(57) ABSTRACT

In various examples, a technique for predicting binding affinity between molecules using a generative artificial intelligence model includes generating, using a generative artificial intelligence model, a first embedding representation of a first molecule and a second embedding representation of a second molecule; calculating a distance in an embedding space between the first embedding representation and the second embedding representation; and predicting, based on the calculated distance in the embedding space, a binding affinity between the first molecule and the second molecule.

### TECHNICAL FIELD

Embodiments of the present disclosure relate generally to computational chemistry, and more specifically to predicting binding affinity based on free-energy-perturbation (FEP) predictions.

### BACKGROUND

In computational chemistry, FEP prediction may be used to predict relative free energy or other thermodynamic deltas between different molecules, ions, or other molecular fragments (e.g., in a biological complex, such as a pairing of a protein and a ligand which may be a small molecule therapeutic, a large molecule therapeutic (e.g., a biologic product), another protein, etc.) without synthesizing molecules and testing the efficacy of synthesized molecules in a therapeutic complex. To predict relative binding free energy (RBFE, also referred to as a relative binding affinity) between two molecules in a biological complex, FEP predictions may be calculated for pairs of molecules from a library of molecules to generate an RBFE prediction for the pair of molecules. The predicted RBFE may subsequently be used to quantify and rank RBFE for a target molecule in a biological complex to determine which molecules are candidates for synthesis and testing for a therapeutic effect relative to the target molecule and which molecules are likely to not have a therapeutic effect relative to the target molecule. For example, molecules with high predicted RBFE may be molecules with a strong bond in a biological complex (e.g., molecules that are ionically bonded, molecules that are bonded based on a large number of shared electrons in a covalent bond, etc.) and thus may be candidates for synthesis and testing. Meanwhile, molecules with low predicted RBFE may have weak bonds in a biological complex (e.g., molecules that are bonded by hydrogen bonds, molecules bonded by van der Waals interaction, etc.) and thus may not be candidates for synthesis and testing.

RBFE predictions may thus be used in the process of developing therapeutics for various diseases by narrowing the search space of target molecules prior to synthesis and real-world testing of such target molecules. While RBFE predictions may allow for the search space of target molecules to be narrowed, the accuracy of these predictions and thus the quality of the target molecules identified based on these RBFE predictions may depend on the structural and energetic differences between different molecules. Generally, for molecules for which a small structural or energetic difference exists, RBFE predictions may be relatively accurate. However, for molecules for which a large structural or energetic difference exists, path interpolation may be used to improve the accuracy of these RBFE predictions because a direct RBFE prediction between very different molecules may be inaccurate.

For each pair of molecules for which a large structural or energetic difference exists, a more accurate RBFE prediction may be made by predicting RBFE between intermediate molecules on a path between the molecules in a pair. Identification of the intermediate molecules may be identified based on scientific principles. While predicting RBFE between intermediate molecules on a path between the molecules in a pair may allow for the prediction of RBFE between molecules in the pair, doing so may be computationally expensive due to the number of paths between molecules in the pair. Further, while errors or inaccuracies in RBFE predictions between intermediate molecules on a path between the molecules in a pair may be relatively small, such errors may accumulate and negatively impact accuracy with an increasing impact as path length (e.g., the number of intermediate molecules in a path between the pair of molecules) increases.

As the foregoing illustrates, what is needed in the art are more effective techniques for predicting relative binding affinity between molecules in a biological complex.

### BRIEF DESCRIPTION OF DRAWINGS

FIG. 1 illustrates a block diagram of a computing system configured to implement one or more aspects of at least one embodiment;

FIG. 2 illustrates an example path diagram from a group of molecules, according to at least one embodiment;

FIG. 3 illustrates example operations for predicting relative binding affinity of molecules in a biological complex based on generative artificial intelligence models;

FIG. 4A illustrates inference and/or training logic, according to at least one embodiment;

FIG. 4B illustrates inference and/or training logic, according to at least one embodiment; and

FIG. 5 illustrates training and deployment of a neural network, according to at least one embodiment.

### DETAILED DESCRIPTION

As discussed herein, identification of molecules for testing as potential treatments for various medical conditions is an important task in drug discovery. By identifying molecules for testing, the process of drug discovery may be accelerated, and resources may be dedicated to testing molecules that are more likely to have a therapeutic effect than those that are less likely to have a therapeutic effect or are likely to have no therapeutic effect at all. As discussed above, however, predictions of relative binding affinity, which correlate to the likelihood of a molecule having a therapeutic effect relative to another molecule, may be inaccurate and may be premised on interpolation of paths for which computation of relative binding affinity is computationally expensive and may be inaccurate. For example, given N candidate molecules, a naïve approach may be to predict the relative binding free energy between each pair of molecules, resulting in

$$\frac{N \times ( N - 1 )}{2}$$

predictions being performed. The number of actual predictions that are performed, however, may scale significantly when the structural and/or energetic difference between different pairs of molecules are large enough that a direct RBFE prediction would be inaccurate. For example, in generating a path from a first molecule to a second molecule, a plurality of intermediate molecules may be designed based on parameter interpolation, maximization of maximum common substructures (MCSs) between successive molecules along a path, or the like. Further, path interpolation techniques that attempt to generate a molecular path from a first molecule to a second molecule may be performed using simulation windows for intermediate A values, with simulation windows being modeled using different intermediate A values for different interaction energy terms in the same window. Path interpolation may be an inefficient process that involves the identification of a path between a first molecule and a second molecule using multiple iterations, each of which may incur a computational expense.

Artificial intelligence models, such as generative artificial intelligence models, allow for the conversion of an input into a point in a high-dimensional latent space, also referred to as an embedding space. Because different molecules, having different structures and energetic properties, these molecules may also be mapped into different embeddings in the embedding space. Thus, embodiments of the present disclosure leverage the ability of generative artificial intelligence models to convert input representations of molecules (e.g., a textual definition, a graph definition, etc.) into points in an embedding space to efficiently and accurately predict a relative binding affinity between molecules in a pair of molecules. Further, when a distance between molecules is sufficiently large, indicating that there is a significant structural and/or energetic difference between molecules, embodiments of the present disclosure may interpolate a path between molecules based on embeddings along a path in the embedding space between embedding representations of the molecules in the pair of molecules.

By using embedding representations of molecules to predict a relative binding affinity between molecules in a pair of molecules, embodiments of the present disclosure may significantly improve the computational efficiency and accuracy of relative binding affinity prediction. A path between molecules in a pair of molecules may be efficiently interpolated based on points in the embedding space along the path between a first molecule and a second molecule in the pair of molecules. Binding affinities between different pairs of intermediate molecules along a path between the first molecule and the second molecule may be predicted, accumulated with other binding affinities for molecules along the path into a total predicted relative binding affinity, and saved for future reference. By doing so, embodiments of the present disclosure may allow for binding affinities to be predicted efficiently based on computationally efficient searches of a database for previously predicted binding affinities between different intermediate molecules. Further, because embedding representations of molecules more accurately reflect chemical and thermodynamic properties of molecules than structural-based mappings that base distance calculations on the largest common structure between molecules, embodiments presented herein may allow for improved accuracy in predicting the relative binding affinity of molecules.

The above examples are not in any way intended to be limiting. As persons skilled in the art will appreciate, as a general matter, the techniques for automatically generating dialogue flows from unlabeled conversation data can be implemented in any suitable application.

The systems and methods described herein may be used for a variety of purposes, by way of example and without limitation, for use in systems associated with machine control, machine locomotion, machine driving, synthetic data generation, model training, perception, augmented reality, virtual reality, mixed reality, robotics, security and surveillance, simulation and digital twinning, autonomous or semi-autonomous machine applications, deep learning, environment simulation, data center processing, conversational AI, generative AI, light transport simulation (e.g., ray-tracing, path tracing, etc.), collaborative content creation for 3D assets, cloud computing and/or any other suitable applications.

Disclosed embodiments may be comprised in a variety of different systems such as automotive systems (e.g., an infotainment or plug-in gaming/streaming system of an autonomous or semi-autonomous machine), systems implemented using a robot, aerial systems, medial systems, boating systems, smart area monitoring systems, systems for performing deep learning operations, systems for performing simulation operations, systems for performing digital twin operations, systems implemented using an edge device, systems incorporating one or more virtual machines (VMs), systems for performing synthetic data generation operations, systems implemented at least partially in a data center, systems for performing conversational AI operations, systems implementing one or more language models-such as large language models (LLMs), small language models (SLMs), vision language models (VLMs), and/or multi-modal language models that may process text, audio, and/or image data, systems for performing light transport simulation, systems for performing collaborative content creation for 3D assets (e.g., systems or platforms that use universal scene descriptor (USD) data, such as OpenUSD), systems implemented at least partially using cloud computing resources, systems for performing generative AI operations, and/or other types of systems.

### System Overview

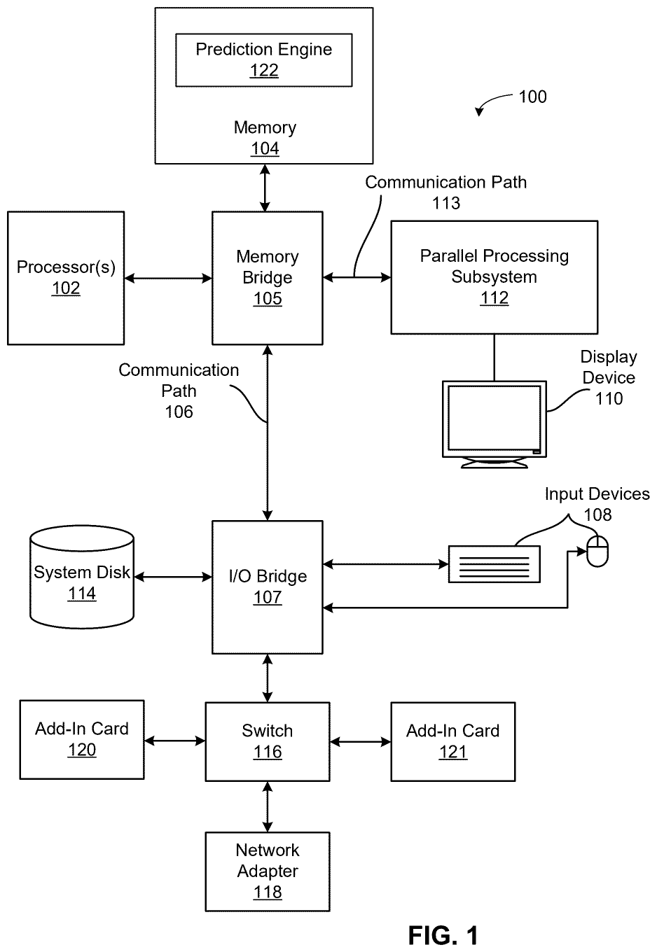

FIG. 1 is a block diagram illustrating a computing system 100 configured to implement one or more aspects of at least one embodiment. In at least one embodiment, computing system 100 may include any type of computing device, including, without limitation, a server machine, a server platform, a desktop machine, a laptop machine, a hand-held/mobile device, a digital kiosk, an in-vehicle infotainment system, a smart speaker or display, a television, and/or a wearable device. In at least one embodiment, computing system 100 is a server machine operating in a data center or a cloud computing environment that provides scalable computing resources as a service over a network.

In various embodiments, computing system 100 includes, without limitation, one or more processors 102 and one or more memories 104 coupled to a parallel processing subsystem 112 via a memory bridge 105 and a communication path 113. Memory bridge 105 is further coupled to an I/O (input/output) bridge 107 via a communication path 106, and I/O bridge 107 is, in turn, coupled to a switch 116.

In one embodiment, I/O bridge 107 is configured to receive user input information from optional input devices 108, such as (but not limited to) a keyboard, mouse, touch screen, sensor data analysis (e.g., evaluating gestures, speech, or other information about one or more uses in a field of view or sensory field of one or more sensors), a VR/MR/AR headset, a gesture recognition system, a steering wheel, mechanical, digital, or touch sensitive buttons or input components, and/or a microphone, and forward the input information to processor(s) 102 for processing. In at least one embodiment, computing system 100 may be a server machine in a cloud computing environment. In such embodiments, computing system 100 may omit input devices 108 and receive equivalent input information as commands (e.g., responsive to one or more inputs from a remote computing device) and/or messages transmitted over a network and received via the network adapter 118. In at least one embodiment, switch 116 is configured to provide connections between I/O bridge 107 and other components of computing system 100, such as a network adapter 118 and various add-in cards 120 and 121.

In at least one embodiment, I/O bridge 107 is coupled to a system disk 114 that may be configured to store content and applications and data for use by processor(s) 102 and parallel processing subsystem 112. In one embodiment, system disk 114 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 107 as well.

In various embodiments, memory bridge 105 may be a Northbridge chip, and I/O bridge 107 may be a Southbridge chip. In addition, communication paths 106 and 113, as well as other communication paths within computing system 100, may be implemented using any technically suitable protocols, including, without limitation, AGP (Accelerated Graphics Port), HyperTransport, or any other bus or point-to-point communication protocol known in the art.

In at least one embodiment, parallel processing subsystem 112 includes a graphics subsystem that delivers pixels to an optional display device 110 that may be any conventional cathode ray tube, liquid crystal display, light-emitting diode display, and/or the like. In such embodiments, parallel processing subsystem 112 may incorporate circuitry optimized for graphics and video processing, including, for example, video output circuitry. Such circuitry may be incorporated across one or more parallel processing units (PPUs), also referred to herein as parallel processors, included within the parallel processing subsystem 112.

In at least one embodiment, parallel processing subsystem 112 incorporates circuitry optimized (e.g., that undergoes optimization) for general purpose and/or compute processing. Again, such circuitry may be incorporated across one or more PPUs included within parallel processing subsystem 112 that are configured to perform such general purpose and/or compute operations. In yet other embodiments, the one or more PPUs included within parallel processing subsystem 112 may be configured to perform graphics processing, general purpose processing, and/or compute processing operations. Memor(ies) 104 include at least one device driver configured to manage the processing operations of the one or more PPUs within parallel processing subsystem 112. In addition, memor(ies) 104 include instructions implementing a prediction engine 122 which can be executed by processor(s) and/or parallel processing subsystem 112.

In various embodiments, parallel processing subsystem 112 may be integrated with one or more of the other elements of FIG. 1 to form a single system. For example, parallel processing subsystem 112 may be integrated with processor(s) 102 and other connection circuitry on a single chip to form a system on a chip (SoC).

Processor(s) 102 may include any suitable processor implemented as a central processing unit (CPU), a graphics processing unit (GPU), an application-specific integrated circuit (ASIC), a field programmable gate array (FPGA), an artificial intelligence (AI) accelerator, a deep learning accelerator (DLA), a parallel processing unit (PPU), a data processing unit (DPU), a vector or vision processing unit (VPU), a programmable vision accelerator (PVA) (which may include one or more VPUs, pixel processing engines (PPEs), and/or direct memory access (DMA) systems), any other type of processing unit, or a combination of different processing units, such as a CPU(s) configured to operate in conjunction with a GPU(s). In general, processor(s) 102 may include any technically feasible hardware unit capable of processing data and/or executing software applications. Further, in the context of this disclosure, the computing elements shown in computing system 100 may correspond to a physical computing system (e.g., a system in a data center or a machine) and/or may correspond to a virtual computing instance executing within a computing cloud.

In at least one embodiment, processor(s) 102 issue commands that control the operation of PPUs. In at least one embodiment, communication path 113 is a Peripheral Component Interconnect Express (PCIe) link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processors 102, and the number of parallel processing subsystems 112, may be modified as desired. For example, in at least one embodiment, memor(ies) 104 may be connected to processor(s) 102 directly rather than through memory bridge 105, and other devices may communicate with memor(ies) 104 via memory bridge 105 and processors 102. In other embodiments, parallel processing subsystem 112 may be connected to I/O bridge 107 or directly to processor(s) 102, rather than to memory bridge 105. In still other embodiments, I/O bridge 107 and memory bridge 105 may be integrated into a single chip instead of existing as one or more discrete devices. In certain embodiments, one or more components shown in FIG. 1 may not be present. For example, switch 116 may be eliminated, and network adapter 118 and add-in cards 120, 121 would connect directly to I/O bridge 107. Further, in certain embodiments, one or more components shown in FIG. 1 may be implemented as virtualized resources in a virtual computing environment, such as a cloud computing environment. In particular, the parallel processing subsystem 112 may be implemented as a virtualized parallel processing subsystem in at least one embodiment. For example, the parallel processing subsystem 112 may be implemented as a virtual graphics processing unit(s) (vGPU(s)) that renders graphics on a virtual machine(s) (VM(s)) executing on a server machine(s) whose GPU(s) and other physical resources are shared across one or more VMs.

Prediction engine 122 generally allows for the use of one or more generative artificial intelligence models to generate embedding representations of molecules for use in predicting the relative binding affinity between a molecule and identification of potential therapeutic compounds for real-world testing.

### Free-Energy-Perturbation (FEP) Path Generation Using Generative Artificial Intelligence Models

To predict the relative binding affinity of molecules (e.g., a different candidate molecules to be bound to a target molecule), embodiments described herein use generative artificial intelligence models executed on prediction engine 122 to generate embedding representations of molecules (e.g., the source or candidate molecule(s), the destination molecule, and intermediate molecule(s)) to determine a distance between molecules (e.g., different candidate molecules) and predict the relative binding affinity between candidate molecules to a destination or target molecule. A generative artificial intelligence model executed on prediction engine 122 may be a molecule foundation model or other artificial intelligence model that can receive an input of a molecule definition and transform the input molecule definition into an embedding representation, which may be a vector in a high-dimensional space. The generative artificial intelligence model may be a model trained to generate these embedding representations from a data set of cheminformatic data such that the embedding space into which inputs are mapped is defined based on cheminformatic principles. The inputs into the generative artificial intelligence model may be, for example, a textual representation of a molecule (e.g., a textual representation of the chemical formula of the molecule), a graph representation of the molecule, or the like.

To predict the relative binding affinity between a first molecule (e.g., a first candidate molecule) and a second molecule (e.g., a second candidate molecule), the generative artificial intelligence model generally generates an embedding representation of the first molecule and an embedding representation of the second molecule in the embedding space. Subsequently a distance (e.g., a Euclidean distance between vectors or embeddings in the embedding space, a Euclidean distance in a space projected from the embedding space, etc.) between the embedding representation of the first molecule and the embedding representation of the second molecule may be calculated to determine the length of a path between the first molecule and the second molecule. The distance may be calculated between the embedding representation of the first molecule and the embedding representation of the second molecule as a value including a direction metric and a quantity metric.

In some embodiments, when the distance in the embedding space between the embedding representation of the first molecule and the embedding representation of the second molecule exceeds a threshold distance, prediction engine 122 can interpolate a path between the first molecule and the second molecule as part of predicting the relative binding affinity between the first molecule and the second molecule. Generally, a path between the first molecule and the second molecule may be a collection of molecules and the associated embedding representations, between the first molecule and the second molecule, and the path may describe a gradual change from the first molecule to the second molecule. To interpolate the path between the first molecule and the second molecule, a plurality of embeddings may be sampled in the space between the embedding representation of the first molecule and the embedding representation of the second molecule. The space along which the embeddings are sampled may be defined, for example, as a space along a shortest path from the embedding representation of the first molecule and the embedding representation of the second molecule. In some embodiments, the plurality of embeddings may be selected based on periodic sampling along the shortest path between the first molecule and the second molecule such that successive molecules represent mildly perturbed versions of previous molecules along the path between the first molecule and the second molecule.

To predict the relative binding affinity between the first molecule and the second molecule, binding affinities may be predicted for each successive pair of molecules along the interpolated path from the first molecule to the second molecule in the embedding space. For example, given a path from a first molecule M₁ to a second molecule M₂, a plurality of intermediate molecules Mᵢ₁-Mᵢₙ may be defined, and the path from the first molecule M₁ to the second molecule M₂ may be defined as M₁, Mᵢ₁, Mᵢ₂, . . . , Mᵢₙ, M₂. A relative binding affinity may be predicted based on the aggregation of predicted binding affinities for successive pairs of molecules along the path from the first molecule M₁ to the second molecule M₂. That is, the predicted relative binding affinity may be calculated as the sum of the binding affinities between M₁ and Mᵢ₁, Mᵢ₁ and Mᵢ₂, and so on, through Mᵢₙ and M₂. Generally, the predicted relative binding affinity between any pair of molecules may be predicted based on various RBFE simulation tools executing on prediction engine 122.

In one example, a path from the first molecule M₁ to the second molecule M₂ may be treated as a direct vector in an embedding space from the first molecule to the second molecule. Prediction engine 122 may perform a linear interpolation along the path to compute N intermediate embeddings between the two embedding representations of the first and second molecules. The N intermediate embeddings may be evenly spaced along the path from the first molecule to the second molecule. Prediction engine 122 can decode the N intermediate embeddings to obtain the N molecules along the path between the first molecule M₁ and the second molecule M₂, with the N molecules forming an optimal path interpolated between the first and second molecules M₁ and M₂. The prediction engine 122 can calculate the relative binding affinity, or relative binding free energy, between the molecules M₁ and M₂ as the sum of relative binding free energies between adjacent molecules along the path between M₁ and M₂ (e.g., based on free energy perturbation (FEP) prediction techniques).

In one example, a path from the first molecule M₁ to the second molecule M₂ may be generated based a graph representation of an input universe of molecules including M₁ and M₂. To build the graph representation of the input universe of molecules, prediction engine 122 constructs a graph with N vertices (corresponding to the N molecules in the input universe of molecules) with edges between each vertex having a weight corresponding to the Euclidean distance between the embedding representations of molecules in the embedding space. To identify a path from M₁ to M₂, prediction engine 122 constructs a minimum spanning tree by selecting N−1 edges in the graph that includes all vertices (molecules) in the input universe of molecules with a minimized sum of weights. Prediction engine 122 can then calculate N−1 relative binding affinities for a specific protein receptor, with each of the N−1 relative binding affinities corresponding to a relative binding affinity for molecules connected by each of the N−1 edges in the minimum spanning tree. Prediction engine 122 can then use the relative binding affinities to derive the full diagram of relative binding free energies between any pair of two molecules, which can be computed as the sum of all edges that bridge the two molecules.

In yet another example, molecule and relative binding affinity information derived from the minimum spanning tree discussed above may be stored in a database for future use. When a new molecule is introduced into the minimum spanning tree, prediction engine 122 can find the molecule in the minimum spanning tree with the closest Euclidean distance to the new molecule. Subsequently, prediction engine 122 calculates the relative binding affinity (or relative binding free energy) to a target protein between the new molecule and the molecule in the minimum spanning tree with the closest Euclidean distance to the new molecule. Relative binding affinities between the new molecule and any other molecule in the minimum spanning tree may be defined as a sum of relative binding affinities along a path from the new molecule to the other molecule in the minimum spanning tree.

Because there is a direct relationship between a textual or graphical representation of a molecule and the embedding representation associated with the molecule, the embedding representations of intermediate molecules identified along an interpolated path may be decoded into the textual or graphical representations of those intermediate molecules. In some embodiments, the resulting intermediate molecules may include molecules progressive modified from the first molecule and/or the second molecule. For example, the intermediate molecule Mᵢ₁ may be a slightly modified version of molecule M₁, the intermediate molecule Mᵢ₂ may be a slightly modified version of molecule Mᵢ₁, and so on. Generally, these progressive modifications to a molecule may correspond to small changes in molecular structure, such as the addition or removal of an atom to a binding point or the like (e.g., a change from a —OMe (methoxy XL) molecule to a —OH (hydroxyl) molecule in which the methyl (CH₃) group bound to an oxygen is changed to a hydrogen atom bound to the oxygen).

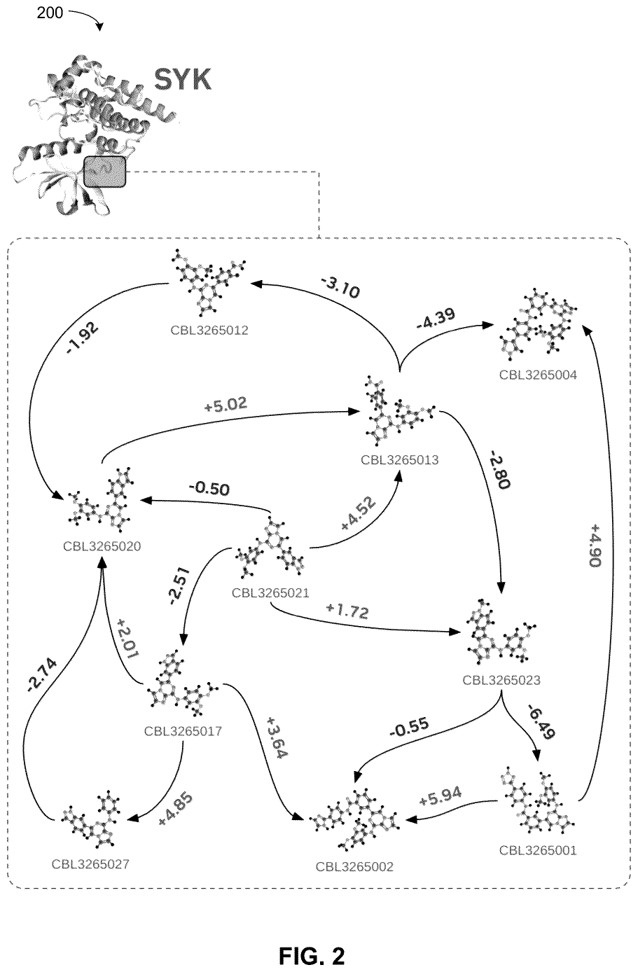

In some embodiments, paths in the embedding space from the first molecule to the second molecule may be represented by a graph, such as the graph 200 illustrated in FIG. 2. In the graph 200, each molecule may be represented by a vertex (or node). Edges between different nodes in the graph 200 generally represent directional paths between different molecules. In some embodiments, a weight associated with each edge in the graph 200 may be associated with the predicted relative binding affinity for the molecules connected by that edge in the graph 200.

To facilitate rapid prediction of the relative binding affinity of molecules in a pair of molecules to a defined target molecule, relative binding affinity information associated with pairs of molecules may be stored in a database for future retrieval. The database may provide a lookup table keyed based on the source molecule and the destination molecule, as relative binding affinity calculations may be dependent on the direction of the interpolated path from the first molecule to the second molecule in the embedding space. When an interpolated path is generated for a pair of molecules, the database can be searched to determine if a relative binding affinity has been predicted for pairs of successive molecules on the interpolated path. If a relative binding affinity has already been predicted for a pair of molecules on the interpolated path, the previously predicted relative binding affinity may be retrieved from the database to minimize redundant computation. In some embodiments, the database may be structured as a hash table in which a hash of the source and destination molecules serves as a key to retrieve a previously-predicted relative binding affinity in O(1) time, a b-tree or other tree data structure that allow for retrieval of a previously-predicted binding affinity in O(log n) time, or the like.

Generally, the predictions of relative binding affinities generated based on embedding representations of molecules generated by an artificial intelligence model may be used by the prediction engine 122 to select molecules for synthesis and testing for a therapeutic effect relative to a target protein. Molecules selected for synthesis may be molecules having the highest predicted binding affinities. In an example in which the techniques described herein are used to calculate relative binding free energy between different candidate molecules for binding to a target protein, the molecules selected for synthesis may be selected based on a relative ordering of molecules, as represented by a traversable data structure in which parent nodes have higher binding affinities than their child nodes. For example, molecules in the top n layers of the traversable data structure may be selected for synthesis. In another example, the predicted binding affinities for different molecules may be used to sort a set of candidate molecules in descending order. The molecules with the top m predicted binding affinities or binding affinities above a threshold binding affinity may be selected by the prediction engine 112 for synthesis and testing.

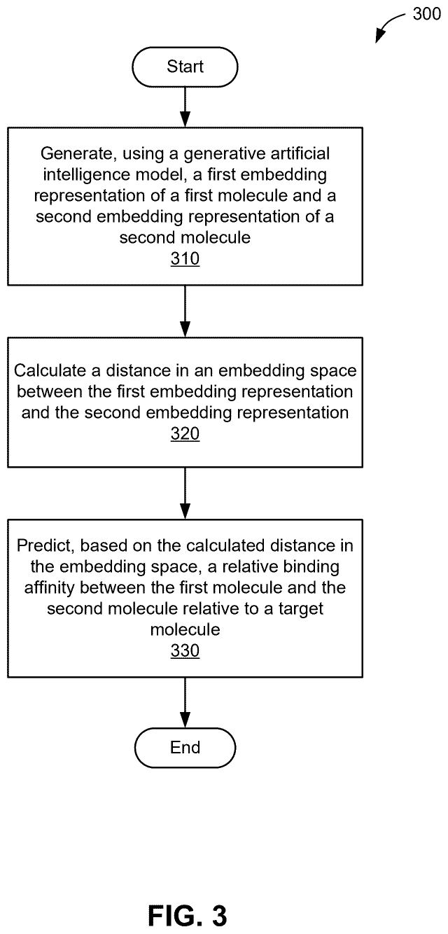

FIG. 3 illustrates example operations 300 for predicting a relative binding affinity between molecules using a generative artificial intelligence model. Operations 300 may be performed, for example, by a computing device, such as a desktop computer, a laptop computer, a server, a cloud computing instance, or the like, on which a prediction engine, such as prediction engine 122 illustrated in FIG. 1, executes.

As illustrated, operations 300 begin at block 310 with generating, using a generative artificial intelligence model, a first embedding representation of a first molecule and a second embedding representation of a second molecule. Generally, the first embedding representation and the second embedding representation are vector representations in a multidimensional embedding space of the first molecule and the second molecule, respectively. These embedding representations may be different from input representations of the first molecule and the second molecule. In some embodiments, the first molecule and the second molecule may be different ligands targeting the same protein to form a protein-ligand complex. The ligands in the protein-ligand complex may be, for example, a small-molecule therapeutic, a biologic, another protein, or other biochemical structure that can bind to the protein.

At block 320, the operations 300 proceed with calculating a distance in an embedding space between the first embedding representation and the second embedding representation.

At block 330, the operations 300 proceed with predicting, based on the calculated distance in the embedding space, a relative binding affinity between the first molecule and the second molecule relative to a target molecule.

In some embodiments, predicting the relative binding affinity between the first molecule and the second molecule may include interpolating a path between the first molecule and the second molecule, the path including a plurality of intermediate molecules. The relative binding affinity between the first molecule and the second molecule may be predicted based on predicted binding affinities between successive intermediate molecules along the interpolated path between the first molecule and the second molecule.

In some embodiments, the plurality of intermediate molecules comprises molecules progressively modified from the first molecule and the second molecule. Generally, progressive modification of molecules may include structural changes to one of the first molecule or the second molecule such that an energetic difference between one intermediate molecule and another intermediate molecule has a relative binding free energy equal to or less than a threshold value (e.g., 1 Kcal/mol, 10 Kcal/mol, etc.).

In some embodiments, interpolating the path between the first molecule and the second molecule may include identifying embeddings in the embedding space along the path between the first molecule and the second molecule and decoding the identified embeddings into the plurality of intermediate molecules. As discussed, within an embedding space, different points in the embedding space may correspond to different molecules. The path interpolated between the first molecule and the second molecule may include a plurality of molecules associated with embeddings along the shortest path in the embedding space between the first molecule and the second molecule. Because a generative artificial intelligence model or other artificial intelligence model can encode data into and decode data out of an embedding space, which may be a multidimensional numerical space representing various features of a molecule, the generative artificial intelligence model can generate textual or graphical representations of intermediate molecules from the corresponding embeddings for these intermediate molecules.

In some embodiments, the operations 300 further include storing, in a database, the plurality of intermediate molecules and the predicted binding affinities between successive intermediate molecules along the interpolated path between the first molecule and the second molecule. The database may be, for example, a hash table, a traversable tree data structure, or other data structure for which a predicted relative binding affinity for a pair of molecules may be efficiently retrieved (e.g., in such a manner that is more computationally efficient than predicting relative binding affinity).

In some embodiments, predicting the relative binding affinity between the first molecule and the second molecule comprises searching a database for predicted relative binding affinities between intermediate molecules along the interpolated path between the first molecule and the second molecule.

In some embodiments, the path between the first molecule and the second molecule is interpolated when the calculated distance in the embedding space exceeds a threshold distance. The threshold distance may correspond to a distance in the embedding space corresponding to a structural or energetic difference between molecules that indicates that two molecules are sufficiently different such that a direct prediction of relative binding affinity may be inaccurate.

In some embodiments, the operations 300 may further include selecting a set of molecules to synthesize based on the relative binding affinity between the first molecule and the second molecule and relative binding affinities between the first molecule and one or more other molecules. For example, the predicted binding affinity may be a relative free binding energy (RBFE) or other metric based on which binding affinity relative to the first molecule can be ranked. In such a case, the second molecule and the one or more other molecules may be sorted based on pairwise relationships between different molecules. For example, the molecules may be ordered in a tree or other traversable data structure where a molecule with a higher binding affinity is located higher in the data structure (e.g., closer to a root node of the tree) than a molecule with a lower binding affinity. In an illustrative example, suppose that molecule A has a higher binding affinity than molecules B and C, and molecule D has a higher binding affinity than molecule B. In such a case, the traversable data structure may place molecules A and D at a first level of the traversable data structure and molecules B and C at a second level of the traversable data structure. Within the traversable data structure, molecule B may have both molecules A and D as parent nodes, while molecule C may have molecule A as a parent node. In another example, the predicted relative binding affinity may be a predicted energy (e.g., measured in calories per mole or other energetic metrics). The molecules (e.g., the second molecule and the one or more other molecules) may be ranked based on the predicted relative binding affinity (e.g., in descending order such that the molecules with the highest binding energy are positioned at the beginning of a data structure and the molecules with the lowest binding energy are positioned at the end of a data structure).

In some embodiments, the selected set of molecules may include a number of molecules selected from the second molecule and the one or more other molecules having highest predicted binding affinities. For example, the selected set of molecules may be the top n molecules in a data structure in which the molecules and binding affinities are organized, molecules in the top m layers of a tree or other traversable data structure, or the like. In some embodiments, the selected set of molecules comprise molecules selected from the second molecule and the one or more other molecules having predicted binding affinities above a threshold binding affinity.

### Inference and Training Logic

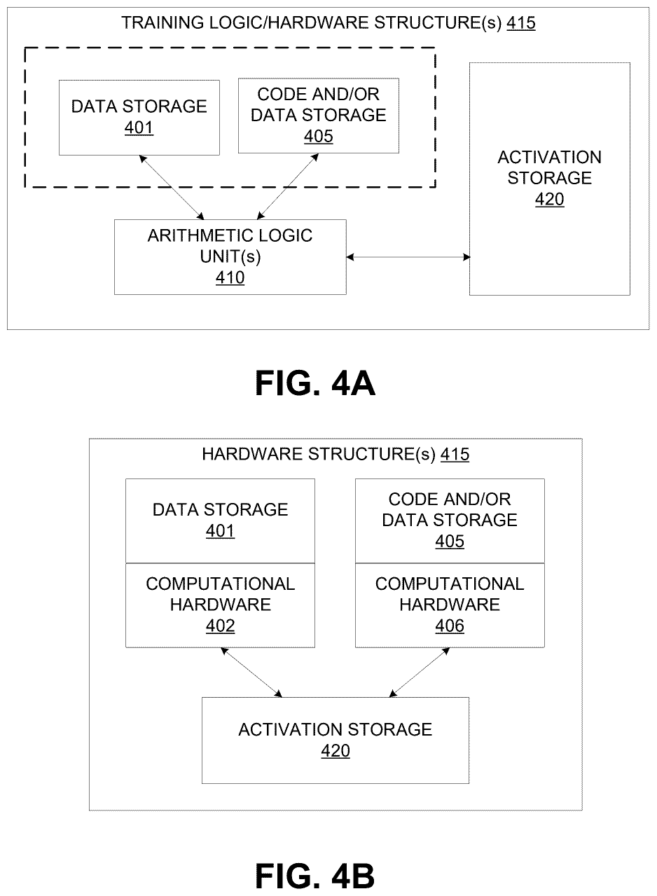

FIG. 4A illustrates inference and/or training logic 415 used to perform inferencing and/or training operations associated with one or more embodiments. Details regarding inference and/or training logic 415 are provided herein in conjunction with at least FIGS. 4A and/or 4B.

In at least one embodiment, inference and/or training logic 415 may include, without limitation, code and/or data storage 401 to store forward and/or output weight and/or input/output data, and/or other parameters to configure neurons or layers of a neural network trained and/or used for inferencing in aspects of one or more embodiments. In at least one embodiment, training logic 415 may include, or be coupled to code and/or data storage 401 to store graph code or other software to control timing and/or order, in which weight and/or other parameter information is to be loaded to configure, logic, including integer and/or floating point units (collectively, arithmetic logic units (ALUs)). In at least one embodiment, code, such as graph code, loads weight or other parameter information into processor ALUs based on an architecture of a neural network to which such code corresponds. In at least one embodiment, code and/or data storage 401 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of code and/or data storage 401 may be included with other on-chip or off-chip data storage, including a processor's L1, L2, or L3 cache or system memory.

In at least one embodiment, any portion of code and/or data storage 401 may be internal or external to one or more processors or other hardware logic devices or circuits. In at least one embodiment, code and/or code and/or data storage 401 may be cache memory, dynamic randomly addressable memory (“DRAM”), static randomly addressable memory (“SRAM”), non-volatile memory (e.g., flash memory), or other storage. In at least one embodiment, a choice of whether code and/or code and/or data storage 401 is internal or external to a processor, for example, or comprising DRAM, SRAM, flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 415 may include, without limitation, a code and/or data storage 405 to store backward and/or output weight and/or input/output data corresponding to neurons or layers of a neural network trained and/or used for inferencing in aspects of one or more embodiments. In at least one embodiment, code and/or data storage 405 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, training logic 415 may include, or be coupled to code and/or data storage 405 to store graph code or other software to control timing and/or order, in which weight and/or other parameter information is to be loaded to configure, logic, including integer and/or floating point units (collectively, arithmetic logic units (ALUs)).

In at least one embodiment, code, such as graph code, causes the loading of weight or other parameter information into processor ALUs based on an architecture of a neural network to which such code corresponds. In at least one embodiment, any portion of code and/or data storage 405 may be included with other on-chip or off-chip data storage, including a processor's L1, L2, or L3 cache or system memory. In at least one embodiment, any portion of code and/or data storage 405 may be internal or external to one or more processors or other hardware logic devices or circuits. In at least one embodiment, code and/or data storage 405 may be cache memory, DRAM, SRAM, non-volatile memory (e.g., flash memory), or other storage. In at least one embodiment, a choice of whether code and/or data storage 405 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, code and/or data storage 401 and code and/or data storage 405 may be separate storage structures. In at least one embodiment, code and/or data storage 401 and code and/or data storage 405 may be a combined storage structure. In at least one embodiment, code and/or data storage 401 and code and/or data storage 405 may be partially combined and partially separate. In at least one embodiment, any portion of code and/or data storage 401 and code and/or data storage 405 may be included with other on-chip or off-chip data storage, including a processor's L1, L2, or L3 cache or system memory.

In at least one embodiment, inference and/or training logic 415 may include, without limitation, one or more arithmetic logic unit(s) (“ALU(s)”) 410, including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage 420 that are functions of input/output and/or weight parameter data stored in code and/or data storage 401 and/or code and/or data storage 405. In at least one embodiment, activations stored in activation storage 420 are generated according to linear algebraic and or matrix-based mathematics performed by ALU(s) 410 in response to performing instructions or other code, wherein weight values stored in code and/or data storage 405 and/or data storage 401 are used as operands along with other values, such as bias values, gradient information, momentum values, or other parameters or hyperparameters, any or all of which may be stored in code and/or data storage 405 or code and/or data storage 401 or another storage on or off-chip.

In at least one embodiment, ALU(s) 410 are included within one or more processors or other hardware logic devices or circuits, whereas in another embodiment, ALU(s) 410 may be external to a processor or other hardware logic device or circuit that uses them (e.g., a coprocessor). In at least one embodiment, ALUs 410 may be included within a processor's execution units or otherwise within a bank of ALUs accessible by a processor's execution units either within same processor or distributed between different processors of different types (e.g., central processing units, graphics processing units, fixed function units, etc.). In at least one embodiment, code and/or data storage 401, code and/or data storage 405, and activation storage 420 may share a processor or other hardware logic device or circuit, whereas in another embodiment, they may be in different processors or other hardware logic devices or circuits, or some combination of same and different processors or other hardware logic devices or circuits. In at least one embodiment, any portion of activation storage 420 may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 420 may be cache memory, DRAM, SRAM, non-volatile memory (e.g., flash memory), or other storage. In at least one embodiment, activation storage 420 may be completely or partially within or external to one or more processors or other logical circuits. In at least one embodiment, a choice of whether activation storage 420 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 415 illustrated in FIG. 4A may be used in conjunction with an application-specific integrated circuit (“ASIC”), such as a TensorFlow® Processing Unit from Google, an inference processing unit (IPU) from Graphcore™, or a Nervana® (e.g., “Lake Crest”) processor from Intel Corp. In at least one embodiment, inference and/or training logic 415 illustrated in FIG. 4A may be used in conjunction with central processing unit (“CPU”) hardware, graphics processing unit (“GPU”) hardware or other hardware, such as field programmable gate arrays (“FPGAs”).

FIG. 4B illustrates inference and/or training logic 415, according to at least one embodiment. In at least one embodiment, inference and/or training logic 415 may include, without limitation, hardware logic in which computational resources are dedicated or otherwise exclusively used in conjunction with weight values or other information corresponding to one or more layers of neurons within a neural network. In at least one embodiment, inference and/or training logic 415 illustrated in FIG. 4B may be used in conjunction with an application-specific integrated circuit (ASIC), such as TensorFlow® Processing Unit from Google, an inference processing unit (IPU) from Graphcore™, or a Nervana® (e.g., “Lake Crest”) processor from Intel Corp. In at least one embodiment, inference and/or training logic 415 illustrated in FIG. 4B may be used in conjunction with central processing unit (CPU) hardware, graphics processing unit (GPU) hardware or other hardware, such as field programmable gate arrays (FPGAs). In at least one embodiment, inference and/or training logic 415 includes, without limitation, code and/or data storage 401 and code and/or data storage 405, which may be used to store code (e.g., graph code), weight values and/or other information, including bias values, gradient information, momentum values, and/or other parameter or hyperparameter information. In at least one embodiment illustrated in FIG. 4B, each of code and/or data storage 401 and code and/or data storage 405 is associated with a dedicated computational resource, such as computational hardware 402 and computational hardware 406, respectively. In at least one embodiment, each of computational hardware 402 and computational hardware 406 comprises one or more ALUs that perform mathematical functions, such as linear algebraic functions, only on information stored in code and/or data storage 401 and code and/or data storage 405, respectively, result of which is stored in activation storage 420.

In at least one embodiment, each of code and/or data storage 401 and 405 and corresponding computational hardware 402 and 406, respectively, correspond to different layers of a neural network, such that resulting activation from one storage/computational pair 401/402 of code and/or data storage 401 and computational hardware 402 is provided as an input to a next storage/computational pair 405/406 of code and/or data storage 405 and computational hardware 406, in order to mirror a conceptual organization of a neural network. In at least one embodiment, each of storage/computational pairs 401/402 and 405/406 may correspond to more than one neural network layer. In at least one embodiment, additional storage/computation pairs (not shown) subsequent to or in parallel with storage/computation pairs 401/402 and 405/406 may be included in inference and/or training logic 415.

### Neural Network Training and Deployment

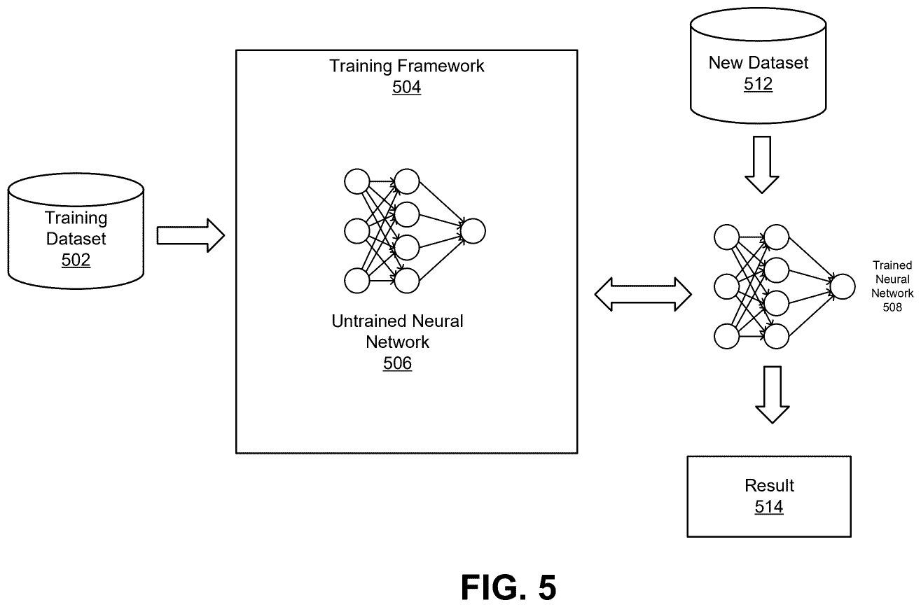

FIG. 5 illustrates training and deployment of a deep neural network, according to at least one embodiment. In at least one embodiment, untrained neural network 506 is trained using a training dataset 502. In at least one embodiment, training framework 504 is a PyTorch framework, whereas in other embodiments, training framework 504 is a TensorFlow, Boost, Caffe, Microsoft Cognitive Toolkit/CNTK, MXNet, Chainer, Keras, Deeplearning4j, or other training framework. In at least one embodiment, training framework 504 trains an untrained neural network 506 and enables it to be trained using processing resources described herein to generate a trained neural network 508. In at least one embodiment, weights may be chosen randomly or by pre-training using a deep belief network. In at least one embodiment, training may be performed in either a supervised, partially supervised, or unsupervised manner.

In at least one embodiment, untrained neural network 506 is trained using supervised learning, wherein training dataset 502 includes an input paired with a desired output for an input, or where training dataset 502 includes input having a known output and an output of neural network 506 is manually graded. In at least one embodiment, untrained neural network 506 is trained in a supervised manner and processes inputs from training dataset 502 and compares resulting outputs against a set of expected or desired outputs. In at least one embodiment, errors are then propagated back through untrained neural network 506. In at least one embodiment, training framework 504 adjusts weights that control untrained neural network 506. In at least one embodiment, training framework 504 includes tools to monitor how well untrained neural network 506 is converging towards a model, such as trained neural network 508, suitable to generating correct answers, such as in result 514, based on input data such as a new dataset 512. In at least one embodiment, training framework 504 trains untrained neural network 506 repeatedly while adjust weights to refine an output of untrained neural network 506 using a loss function and adjustment algorithm, such as stochastic gradient descent. In at least one embodiment, training framework 504 trains untrained neural network 506 until untrained neural network 506 achieves a desired accuracy. In at least one embodiment, trained neural network 508 can then be deployed to implement any number of machine learning operations.

In at least one embodiment, untrained neural network 506 is trained using unsupervised learning, wherein untrained neural network 506 attempts to train itself using unlabeled data. In at least one embodiment, unsupervised learning training dataset 502 will include input data without any associated output data or “ground truth” data. In at least one embodiment, untrained neural network 506 can learn groupings within training dataset 502 and can determine how individual inputs are related to untrained dataset 502. In at least one embodiment, unsupervised training can be used to generate a self-organizing map in trained neural network 508 capable of performing operations useful in reducing dimensionality of new dataset 512. In at least one embodiment, unsupervised training can also be used to perform anomaly detection, which allows identification of data points in new dataset 512 that deviate from normal patterns of new dataset 512.

In at least one embodiment, semi-supervised learning may be used, which is a technique in which in training dataset 502 includes a mix of labeled and unlabeled data. In at least one embodiment, training framework 504 may be used to perform incremental learning, such as through transferred learning techniques. In at least one embodiment, incremental learning enables trained neural network 508 to adapt to new dataset 512 without forgetting knowledge instilled within trained neural network 508 during initial training.

In at least one embodiment, training framework 504 is a framework processed in connection with a software development toolkit such as an OpenVINO (Open Visual Inference and Neural network Optimization) toolkit. In at least one embodiment, an OpenVINO toolkit is a toolkit such as those developed by Intel Corporation of Santa Clara, CA.

In at least one embodiment, OpenVINO is a toolkit for facilitating development of applications, specifically neural network applications, for various tasks and operations, such as human vision emulation, speech recognition, natural language processing, recommendation systems, and/or variations thereof. In at least one embodiment, OpenVINO supports neural networks such as convolutional neural networks (CNNs), recurrent and/or attention-based neural networks, and/or various other neural network models. In at least one embodiment, Open VINO supports various software libraries such as OpenCV, OpenCL, and/or variations thereof.

In at least one embodiment, OpenVINO supports neural network models for various tasks and operations, such as classification, segmentation, object detection, face recognition, speech recognition, pose estimation (e.g., humans and/or objects), monocular depth estimation, image inpainting, style transfer, action recognition, colorization, and/or variations thereof.

In at least one embodiment, OpenVINO comprises one or more software tools and/or modules for model optimization, also referred to as a model optimizer. In at least one embodiment, a model optimizer is a command line tool that facilitates transitions between training and deployment of neural network models. In at least one embodiment, a model optimizer optimizes neural network models for execution on various devices and/or processing units, such as a GPU, CPU, PPU, GPGPU, and/or variations thereof. In at least one embodiment, a model optimizer generates an internal representation of a model, and optimizes said model to generate an intermediate representation. In at least one embodiment, a model optimizer reduces a number of layers of a model. In at least one embodiment, a model optimizer removes layers of a model that are utilized for training. In at least one embodiment, a model optimizer performs various neural network operations, such as modifying inputs to a model (e.g., resizing inputs to a model), modifying a size of inputs of a model (e.g., modifying a batch size of a model), modifying a model structure (e.g., modifying layers of a model), normalization, standardization, quantization (e.g., converting weights of a model from a first representation, such as floating point, to a second representation, such as integer), and/or variations thereof.

In at least one embodiment, Open VINO comprises one or more software libraries for inferencing, also referred to as an inference engine. In at least one embodiment, an inference engine is a C++ library, or any suitable programming language library. In at least one embodiment, an inference engine is utilized to infer input data. In at least one embodiment, an inference engine implements various classes to infer input data and generate one or more results. In at least one embodiment, an inference engine implements one or more API functions to process an intermediate representation, set input and/or output formats, and/or execute a model on one or more devices.

In at least one embodiment, OpenVINO provides various abilities for heterogeneous execution of one or more neural network models. In at least one embodiment, heterogeneous execution, or heterogeneous computing, refers to one or more computing processes and/or systems that utilize one or more types of processors and/or cores. In at least one embodiment, Open VINO provides various software functions to execute a program on one or more devices. In at least one embodiment, OpenVINO provides various software functions to execute a program and/or portions of a program on different devices. In at least one embodiment, Open VINO provides various software functions to, for example, run a first portion of code on a CPU and a second portion of code on a GPU and/or FPGA. In at least one embodiment, Open VINO provides various software functions to execute one or more layers of a neural network on one or more devices (e.g., a first set of layers on a first device, such as a GPU, and a second set of layers on a second device, such as a CPU).

In at least one embodiment, OpenVINO includes various functionality similar to functionalities associated with a CUDA programming model, such as various neural network model operations associated with frameworks such as TensorFlow, PyTorch, and/or variations thereof. In at least one embodiment, one or more CUDA programming model operations are performed using OpenVINO. In at least one embodiment, various systems, methods, and/or techniques described herein are implemented using OpenVINO.

Other variations are within spirit of present disclosure. Thus, while disclosed techniques are susceptible to various modifications and alternative constructions, certain illustrated embodiments thereof are shown in drawings and have been described herein in detail. It should be understood, however, that there is no intention to limit disclosure to specific form or forms disclosed, but on contrary, intention is to cover all modifications, alternative constructions, and equivalents falling within spirit and scope of disclosure, as defined in appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. “Connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. In at least one embodiment, use of term “set” (e.g., “a set of items”) or “subset” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, term “plurality” indicates a state of being plural (e.g., “a plurality of items” indicates multiple items). In at least one embodiment, number of items in a plurality is at least two, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. In at least one embodiment, set of non-transitory computer-readable storage media comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors—for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

In at least one embodiment, an arithmetic logic unit is a set of combinational logic circuitry that takes one or more inputs to produce a result. In at least one embodiment, an arithmetic logic unit is used by a processor to implement mathematical operation such as addition, subtraction, or multiplication. In at least one embodiment, an arithmetic logic unit is used to implement logical operations such as logical AND/OR or XOR. In at least one embodiment, an arithmetic logic unit is stateless, and made from physical switching components such as semiconductor transistors arranged to form logical gates. In at least one embodiment, an arithmetic logic unit may operate internally as a stateful logic circuit with an associated clock. In at least one embodiment, an arithmetic logic unit may be constructed as an asynchronous logic circuit with an internal state not maintained in an associated register set. In at least one embodiment, an arithmetic logic unit is used by a processor to combine operands stored in one or more registers of the processor and produce an output that can be stored by the processor in another register or a memory location.

In at least one embodiment, as a result of processing an instruction retrieved by the processor, the processor presents one or more inputs or operands to an arithmetic logic unit, causing the arithmetic logic unit to produce a result based at least in part on an instruction code provided to inputs of the arithmetic logic unit. In at least one embodiment, the instruction codes provided by the processor to the ALU are based at least in part on the instruction executed by the processor. In at least one embodiment combinational logic in the ALU processes the inputs and produces an output which is placed on a bus within the processor. In at least one embodiment, the processor selects a destination register, memory location, output device, or output storage location on the output bus so that clocking the processor causes the results produced by the ALU to be sent to the desired location.

In the scope of this application, the term arithmetic logic unit, or ALU, is used to refer to any computational logic circuit that processes operands to produce a result. For example, in the present document, the term ALU can refer to a floating point unit, a DSP, a tensor core, a shader core, a coprocessor, or a CPU.

Accordingly, in at least one embodiment, computer systems are configured to implement one or more services that singly or collectively perform operations of processes described herein and such computer systems are configured with applicable hardware and/or software that enable performance of operations. Further, a computer system that implements at least one embodiment of present disclosure is a single device and, in another embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of disclosure and does not pose a limitation on scope of disclosure unless otherwise claimed. No language in specification should be construed as indicating any non-claimed element as essential to practice of disclosure.

All references, including publications, patent applications, and patents, cited herein are hereby incorporated by reference to same extent as if each reference were individually and specifically indicated to be incorporated by reference and were set forth in its entirety herein.

In description and claims, terms “coupled” and “connected,” along with their derivatives, may be used. It should be understood that these terms may be not intended as synonyms for each other. Rather, in particular examples, “connected” or “coupled” may be used to indicate that two or more elements are in direct or indirect physical or electrical contact with each other. “Coupled” may also mean that two or more elements are not in direct contact with each other, but yet still co-operate or interact with each other.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. In at least one embodiment, terms “system” and “method” are used herein interchangeably insofar as system may embody one or more methods and methods may be considered a system.

In present document, references may be made to obtaining, acquiring, receiving, or inputting analog or digital data into a subsystem, computer system, or computer-implemented machine. In at least one embodiment, process of obtaining, acquiring, receiving, or inputting analog and digital data can be accomplished in a variety of ways such as by receiving data as a parameter of a function call or a call to an application programming interface. In at least one embodiment, processes of obtaining, acquiring, receiving, or inputting analog or digital data can be accomplished by transferring data via a serial or parallel interface. In at least one embodiment, processes of obtaining, acquiring, receiving, or inputting analog or digital data can be accomplished by transferring data via a computer network from providing entity to acquiring entity. In at least one embodiment, references may also be made to providing, outputting, transmitting, sending, or presenting analog or digital data. In various examples, processes of providing, outputting, transmitting, sending, or presenting analog or digital data can be accomplished by transferring data as an input or output parameter of a function call, a parameter of an application programming interface or interprocess communication mechanism.

Although descriptions herein set forth example implementations of described techniques, other architectures may be used to implement described functionality, and are intended to be within scope of this disclosure. Furthermore, although specific distributions of responsibilities may be defined above for purposes of description, various functions and responsibilities might be distributed and divided in different ways, depending on circumstances.

### EXAMPLE CLAUSES

Implementation details of various aspects of the present disclosure may be found in the following numbered clauses:

1. In some embodiments, a method comprises generating, using a generative artificial intelligence model, a first embedding representation of a first molecule and a second embedding representation of a second molecule; calculating a distance in an embedding space between the first embedding representation and the second embedding representation; and predicting, based on the calculated distance in the embedding space, a relative binding affinity between the first molecule and the second molecule.

2. The method of clause 1, further comprising: selecting a set of molecules to synthesize based on the relative binding affinity between the first molecule and the second molecule and binding affinities between the first molecule and one or more other molecules.

3. The method of clause 2, wherein the selected set of molecules comprise a number of molecules selected from the second molecule and the one or more other molecules having highest predicted binding affinities.

4. The method of any of clauses 2 or 3, wherein the selected set of molecules comprise molecules selected from the second molecule and the one or more other molecules having predicted binding affinities above a threshold binding affinity.

5. The method of any of clauses 1 through 4, wherein predicting the relative binding affinity between the first molecule and the second molecule comprises: interpolating a path between the first molecule and the second molecule, the path including a plurality of intermediate molecules; and predicting the relative binding affinity between the first molecule and the second molecule based on predicted binding affinities between successive intermediate molecules along the interpolated path between the first molecule and the second molecule.

6. The method of clause 5, wherein the plurality of intermediate molecules comprises molecules progressively modified from the first molecule and the second molecule.

7. The method of any of clauses 5 or 6, wherein interpolating the path between the first molecule and the second molecule comprises: identifying embeddings in the embedding space along the path between the first molecule and the second molecule; and decoding the identified embeddings into the plurality of intermediate molecules.

8. The method of clause 7, further comprising storing, in a database, the plurality of intermediate molecules and the predicted binding affinities between successive intermediate molecules along the interpolated path between the first molecule and the second molecule.

9. The method of any of clauses 5 through 8, wherein predicting the relative binding affinity between the first molecule and the second molecule comprises searching a database for predicted binding affinities between intermediate molecules along the interpolated path between the first molecule and the second molecule.

10. The method of any of clauses 5 through 9, wherein the path between the first molecule and the second molecule is interpolated when the calculated distance in the embedding space exceeds a threshold distance.

11. The method of any of clauses 1 through 10, wherein the first molecule comprises a first ligand targeting a protein in a protein-ligand complex and the second molecule comprises a second ligand targeting the protein.

12. A processing system, comprising: at least one memory having executable instructions stored thereon; and one or more processors configured to execute the executable instructions to cause the processing system to perform the operations of any of clauses 1 through 11.

13. A processing system, comprising: means for performing the operations of any of clauses 1 through 11.

14. A non-transitory computer-readable medium having executable instructions stored thereon which, when executed by one or more processors, performs the operations of any of clauses 1 through 11.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing the claims.

What is claimed is:

1. A processor-implemented method, comprising:
generating, using a generative artificial intelligence model, a first embedding representation of a first molecule and a second embedding representation of a second molecule, the first embedding representation and the second embedding representation comprising vector representations in a multidimensional embedding space of the first molecule and the second molecule, respectively, different from an input representation of the first molecule and the second molecule;
calculating a distance in the embedding space between the first embedding representation and the second embedding representation; and
predicting, based on the calculated distance in the embedding space, a relative binding affinity between the first molecule and the second molecule relative to a target molecule.

2. The method of claim 1, further comprising:
selecting a set of molecules to synthesize based on the relative binding affinity between the first molecule and the second molecule and relative binding affinities between the first molecule and one or more other molecules.

3. The method of claim 2, wherein the selected set of molecules comprise a number of molecules selected from the second molecule and the one or more other molecules having highest predicted binding affinities.

4. The method of claim 2, wherein the selected set of molecules comprise molecules selected from the second molecule and the one or more other molecules having predicted binding affinities above a threshold binding affinity.

5. The method of claim 1, wherein predicting the relative binding affinity between the first molecule and the second molecule comprises:
interpolating a path between the first molecule and the second molecule, the path including a plurality of intermediate molecules; and
predicting the relative binding affinity between the first molecule and the second molecule based on predicted relative binding affinities between successive intermediate molecules along the interpolated path between the first molecule and the second molecule.

6. The method of claim 5, wherein the plurality of intermediate molecules comprises molecules progressively modified from the first molecule and the second molecule.

7. The method of claim 5, wherein interpolating the path between the first molecule and the second molecule comprises:
identifying embeddings in the embedding space along the path between the first molecule and the second molecule; and
decoding the identified embeddings into the plurality of intermediate molecules.

8. The method of claim 7, further comprising storing, in a database, the plurality of intermediate molecules and the predicted relative binding affinities between successive intermediate molecules along the interpolated path between the first molecule and the second molecule.

9. The method of claim 5, wherein predicting the relative binding affinity between the first molecule and the second molecule comprises searching a database for predicted relative binding affinities between intermediate molecules along the interpolated path between the first molecule and the second molecule.

10. The method of claim 5, wherein the path between the first molecule and the second molecule is interpolated when the calculated distance in the embedding space exceeds a threshold distance.

11. The method of claim 1, wherein the first molecule comprises a first ligand targeting a protein in a protein-ligand complex and the second molecule comprises a second ligand targeting the protein.

12. A processing system, comprising:
at least one memory having executable instructions stored thereon; and
one or more processors configured to execute the executable instructions to cause the processing system to:
generate, using a generative artificial intelligence model, a first embedding representation of a first molecule and a second embedding representation of a second molecule, the first embedding representation and the second embedding representation comprising vector representations in a multidimensional embedding space of the first molecule and the second molecule, respectively, different from an input representation of the first molecule and the second molecule;
calculate a distance in the embedding space between the first embedding representation and the second embedding representation; and
predict, based on the calculated distance in the embedding space, a relative binding affinity between the first molecule and the second molecule relative to a target molecule.

13. The processing system of claim 12, wherein the one or more processors are further configured to cause the processing system to:
select a set of molecules to synthesize based on the relative binding affinity between the first molecule and the second molecule and relative binding affinities between the first molecule and one or more other molecules.

14. The processing system of claim 12, wherein to predict the relative binding affinity between the first molecule and the second molecule, the one or more processors are configured to cause the processing system to:
interpolate a path between the first molecule and the second molecule, the path including a plurality of intermediate molecules; and
predict the relative binding affinity between the first molecule and the second molecule based on predicted relative binding affinities between successive intermediate molecules along the interpolated path between the first molecule and the second molecule.

15. The processing system of claim 14, wherein the plurality of intermediate molecules comprises molecules progressively modified from the first molecule and the second molecule.

16. The processing system of claim 14, wherein to interpolate the path between the first molecule and the second molecule, the one or more processors are configured to cause the processing system to:
identify embeddings in the embedding space along the path between the first molecule and the second molecule; and
decode the identified embeddings into the plurality of intermediate molecules.

17. The processing system of claim 16, wherein the one or more processors are configured to cause the processing system to store, in a database, the plurality of intermediate molecules and the predicted relative binding affinities between successive intermediate molecules along the interpolated path between the first molecule and the second molecule.

18. The processing system of claim 14, wherein to predict the relative binding affinity between the first molecule and the second molecule, the one or more processors are configured to cause the processing system to search a database for predicted relative binding affinities between intermediate molecules along the interpolated path between the first molecule and the second molecule.

19. The processing system of claim 14, wherein the path between the first molecule and the second molecule is interpolated when the calculated distance in the embedding space exceeds a threshold distance.

20. The processing system of claim 12, wherein the system is comprised in at least one of:
a system for performing simulation operations;
a system for performing digital twin operations;
a system for performing collaborative content creation for 3D assets;
a system for performing one or more deep learning operations;
a system implemented using an edge device;
a system for generating or presenting at least one of virtual reality content, augmented reality content, or mixed reality content;
a system implemented using a robot;
a system for performing one or more conversational AI operations;
a system implemented using one or more large language models (LLMs);
a system implemented using one or more small language models (SLMs);
a system implementing one or more vision language models (VLMs);
a system implementing one or more multi modal language models;
a system for generating synthetic data;
a system for performing one or more generative AI operations;
a system incorporating one or more virtual machines (VMs);
a system implemented at least partially in a data center; or
a system implemented at least partially using cloud computing resources.
